(19) United States
(12) Patent Application Publication
(10) Pub. No.: US 2011/0182769 A1
(43) Pub. Date: Jul. 28, 2011

(54) SANITIZING DEVICE FOR A SHOPPING CART HANDLE

(76) Inventor: David Wayne Rich, Marietta, GA (US)

(21) Appl. No.: 12/909,485

(22) Filed: Oct. 21, 2010

Related U.S. Application Data
(60) Provisional application No. 61/298,850, filed on Jan. 27, 2010.

(51) Int. Cl. A61L 2/18

(52) U.S. Cl. 422/28; 422/292

(57) ABSTRACT

A sanitizing device for a handle bar is provided. The device has an elongated axis. The device comprises a housing unit for mounting on the handle bar with the housing fitting around the handle bar. The device also has a reservoir inside the housing unit for storing sanitizing materials and a dispenser inside the housing unit. The dispenser connected to the reservoir for dispensing sanitizing materials from the reservoir to the handle bar

### CROSS REFERENCE TO RELATED PATENT APPLICATIONS

The instant patent application herein claims priority to U.S. Provisional Patent Application No. 61/298,850 entitled “A Sanitizing Device for a Shopping Cart Handle” by David Wayne Rich filed on Jan. 27, 2010, which is herein incorporated by reference in its entirety.

### FIELD OF THE INVENTION

The present disclosure is directed to a device to sanitize a handle bar. More particularly, the present disclosure is directed to a device that may rest on a handle bar that can move relative to the handle bar and to apply a disinfectant to a handle, for example, a shopping cart handle.

### BACKGROUND OF THE RELATED ART

Shopping carts are known in the art. Generally, the car comprises a bin connected to one or more wheels. A handle is provided so a user may push the cart around the store and place the items desired in the bin. Generally, the shopping carts can be used extensively both inside and outside of the store and sometimes are left in the parking lot of the store. Generally, the shopping carts can be very dirty from so many people shopping and touching the handle and the people pushing the carts around. Moisture, dirt and foreign matter can accumulate on the handle and on the bins. Generally, the shopping carts and handle are rarely washed in some establishments. Generally, viruses, bacteria and pathogens can also be transmitted from user to user from the shopping cart handle, which is disfavored. Some users use cleaning wipes or cleaning products. Products or the wipes are applied on the handle or on the user's hands to clean the handle and kill any viruses, bacteria or pathogens from the handle. However, in the event that the user runs out of the cleaning wipes or cleaning products, generally there is little recourse for the user, and many users simply use the dirty shopping cart without cleaning thereby putting the users at risk for contamination and possible sickness.

### SUMMARY OF THE INVENTION

It is an object of the present invention to provide a shopping cart handle that can be quickly and easily cleaned. It is also an object of the present invention to provide a device that can quickly and easily clean the handle without touching the cleaning fluid or touching the handle. It is also an object of the present invention to provide a device that dispenses a cleaning fluid from an inner surface of the device. It is also an object of the present invention to provide a cleaning device that completely surrounds the shopping cart handle. It is also an object of the present invention to provide a cleaning device that fits on the handle and can be left on the cart's handle in a permanent fashion for each user. It is also an object of the present invention to provide a cleaning device that dispenses a cleaning fluid from inside the device. It is also an object of the present invention to provide a cleaning device that has a receptacle for storing a cleaning fluid. It is also an object of the present invention to provide a cleaning device that fits on the handle and that includes a squeegee material on the device. It is also an object of the present invention to provide a cleaning device that dispenses a fluid that can evaporate quickly.

According to a first aspect of the present disclosure, there is provided a sanitizing device for a handle bar. The device has an elongated axis. The device comprises a housing unit for mounting on the handle bar with the housing fitting around the handle bar. The device also has a reservoir inside the housing unit for storing sanitizing materials and a dispenser inside the housing unit. The dispenser connected to the reservoir for dispensing sanitizing materials from the reservoir to the handle bar.

In yet another aspect of the present disclosure there is provided a method of cleaning a handle bar comprising fitting a device around the handle bar and dispensing a material from the device to the handle bar and moving the device on the handle bar with a second material connected to the device to clean the handle bar with the second material.

In another embodiment of the present disclosure, there is provided a cleaning device for a handle bar comprising a housing completely surrounding the handle bar and a nozzle for dispensing a cleaning fluid to the handle bar with the nozzle located within the housing. The housing comprises a first aperture and a second aperture and the first aperture and the second aperture are on opposite lateral sides of the housing. A squeegee material surrounds the first aperture and the second aperture.

### BRIEF DESCRIPTION OF THE FIGURES

The foregoing and other objects, features and advantages of the invention will be apparent from the following more particular description of preferred embodiments of the invention, as illustrated in the accompanying drawings in which like reference characters refer to the same parts throughout different views. The drawings are not meant to limit the invention to particular mechanisms for carrying out the invention in practice, but rather, the drawings are illustrative of certain ways of performing the invention. Others will be readily apparent to those skilled in the art.

FIG. 1 is a perspective view of the sanitizing device mounted to a handle bar illustrating one exemplary embodiment;

FIG. 2 is a close up perspective view of the sanitizing device mounted to the handle bar of FIG. 1;

FIG. 3 is a perspective view of a lower housing portion of the sanitizing device with the upper housing portion being removed for illustration shown a first and a second slide rails;

FIG. 4 is an exploded view of the lower housing portion of FIG. 3 illustrating the first and second slide rails, pump, first and second apertures, curved support surface, drain holes and other components of the sanitizing device;

FIGS. 5 and 6 show a front and side view of the lower housing portion of FIG. 3;

FIG. 7 shows a cross sectional view of the lower housing portion of FIG. 3;

FIG. 8 shows a top view of the lower housing portion of FIG. 3;

FIG. 9A shows a bottom perspective view of the upper housing portion showing a tubular member for depositing the fluid on the handle bar;

FIG. 9B shows a bottom view of the upper housing portion showing the tubular member for depositing the fluid on the handle bar;

FIGS. 10 and 11 show respective front and side views of the upper housing portion with a pump arm extending from a bottom side thereof;

FIG. 12 shows an exploded view of the upper housing and the components therein; and

FIGS. 13 and 14 show a top and a front view of the sanitizing device being moved relative to the stationary handle bar for cleaning the handle bar.

### DETAILED DESCRIPTION OF THE PREFERRED EMBODIMENTS

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention generally provides a sanitizing device. More specifically, the current invention provides a sanitizing device for a shopping cart handle.

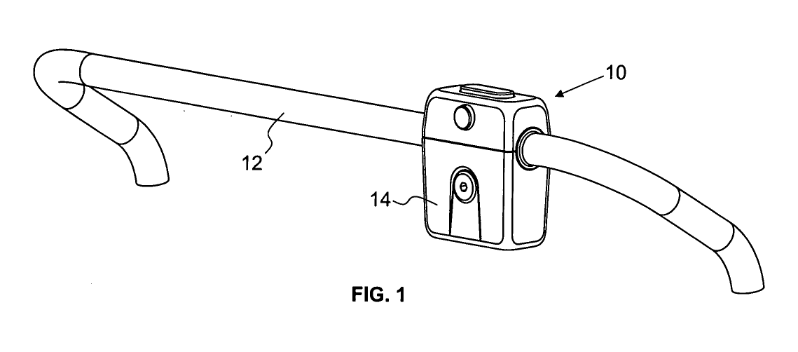

Turning now to FIG. 1, there is shown a sanitizing device 10 for a handle bar 12. Preferably, the sanitizing device 10 can move laterally relative to the handle bar 12 to clean the handle bar 12 with a fluid or disinfectant or the like. Preferably, the sanitizing device 10 sweeps back and forth along the handle bar 12 to release a chemical disinfectant as the device 10 passes along a top surface of the handle bar 12. Preferably, the disinfectant covers the handle bar 12 and outer surface thereof. After a period of time, the disinfectant evaporates and kills the germs and pathogens located on the handle bar 12. Thereafter, the sanitizing device 10 may be put off to one side and the handle 12 can be free to touch and hold and use, for example, on a supermarket cart, or on a baby carriage or the like. In one embodiment, the device 10 can be pushed relative to the handle 12. In another alternative embodiment, the device 10 may be motorized and move without being pushed and actuated based on motion, a signal, or an input button or the like.

The sanitizing device 10 of FIG. 1 preferably has an elongated axis, which includes a housing unit 14, a reservoir (not shown), and a dispenser (not shown). The housing unit 14 may be mounted and fitted on a handle bar 12 and preferably can be removably connected to the handle bar 12 and placed there by a user and then removed or can be permanently connected to the handle bar 12. Inside the housing unit 14, there may be a reservoir for storing sanitizing materials. In addition, a dispenser, inside the housing unit 14, may be connected to the reservoir for dispensing sanitizing materials from the reservoir to the handle bar 12. More specifically, housing 14 may have two parts to wrap around a handle bar 12, such as a shopping cart handle bar 12. A reservoir inside the housing unit may be filled with sanitizing materials, such as sanitizing fluid. There may be small brushes and squeegees inside the housing unit 14. The squeegees may be designed to help drying of the handle bar 12. Furthermore, the current device 10 may have an easy refill locked cap.

The current invention may have various designs, such as a round style or an envelope style. The device 10 also may have an operational gauge for fluid. In addition, the current device 10 may be made of polyvinylchloride (PVC) or aluminum. In one non-limiting embodiment of the present disclosure, the device 10 body can be 4″×2″×5″ but may have various different dimensions. In addition, the invention may have safety features. For example, it may be difficult for children and customers to open it, but it may be easy for professionals to open for services and refills. A user may swipe back and forth the current device 10 to disinfect a handle bar 12.

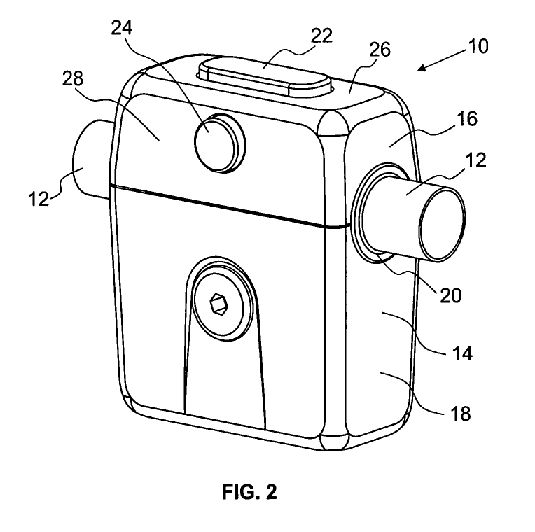

Turning now to FIG. 2, there is shown a close up perspective view of the sanitizing device 10 riding on a handle 12, which is segmented for illustration. As can be seen from FIG. 2, the sanitizing device 10 includes a housing 14, which is a resilient member to house internal components therein. Preferably, the housing 14 is formed from any resilient member known in the art that is lightweight and that is strong for repeated use and that can withstand the forces of a user gripping and sliding the housing 14 relative to the handle bar 12. Preferably, the housing 14 comprises at least two portions or an upper housing 16 and a lower housing 18.

Preferably, the upper housing 16 is detachably connected to the lower housing 18 so the upper housing 16 can be removably disconnected from the lower housing 18 to reveal the interior components of the housing 14. Preferably, the upper housing 16 and the lower housing 18 when connected form a rectangular shaped member in cross section; however, this shape forms no limitation to the present disclosure and the housing 14 may have various shapes known in the art such as oblong, spherical or the like. Preferably, the lateral side of the housing 14 comprises at least two apertures 20. The apertures 20 permit the handle bar 12 access through the housing 14. Preferably, the apertures 20 are circular shaped and generally conform to the cross sectional shape of the handle bar 12. If the handle bar 12 was generally square shaped in a cross sectional view, then the apertures 20 likewise would conform to the handle bar 12 and so on.

The housing 14 further comprises at least two inputs 22 and 24 disposed on the housing 14. Preferably, a first input or button 22 is disposed on a top surface 26 of the housing 14. Preferably, a second input or button 24 is disposed on a lateral surface 28 of the housing 14. It should be appreciated that at least one button 22 or 24 provides an unlocking feature or command to separate the upper housing and the lower housing 16 and 18. It should also be appreciated that at least one button 22 or 24 provides a second feature or a release of the disinfectant or solution from the housing 14 as will be discussed herein to be applied to the handle bar 12. It should be appreciated that the buttons 22 and 24 may be placed in different locations of the housing 14, or that the functions of the buttons may be inverted with the button 24 providing the unlocking of the housing 14 and the first button 22 providing the solution to be released from the device 10. In an alternative embodiment, the buttons 22 and 24 may be placed on the lower housing 18.

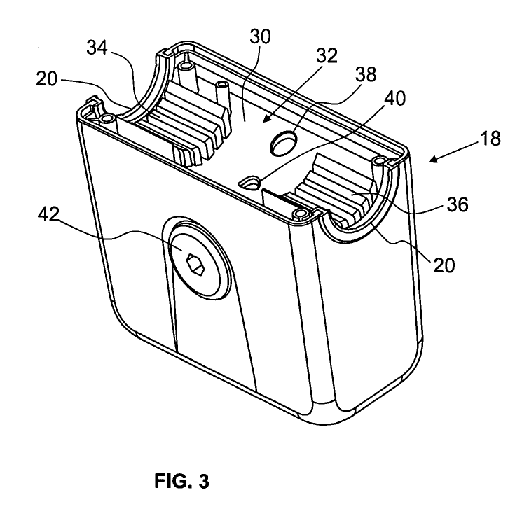

Turning now to FIG. 3, there is shown a bottom view of the lower housing 18 of the sanitizing device 10 with the upper housing 16 being removed for illustration purposes. As can be seen, the apertures 20 are revealed on the lateral sides to allow the bar 12 access to an interior space 32 of the lower housing 18. The interior space 32 preferably includes a curved interior surface 30 that is within lower housing 18 and in the interior space 32.

The curved interior surface 30 preferably complements the curvature of the handle bar 12. This curvature allows the curved interior surface 30 to traverse over and ride the handle bar 12 as the lower housing 18 is moved laterally relative to the handle bar 12. The curved interior surface 30 preferably comprises a first and a second slide rails 34 and 36 that are disposed on the curved interior surface 30 and also within the interior space 32.

The first and the second slide rails 34 and 36 preferably are curved members that fit on the handle bar 12 and traverse over the handle bar 12. The first and second slide rails 34 and 36 preferably form a way track or, more particularly a track or surface structure that supports and guides the device 10 on the handle bar 12.

Below the first and the second slide rails 34 and 36 is a formation that improves ground stability of the device 10 and provides drainage (apertures 40 and 38) as the device 10 moves across the handle bar 12. Preferably, the curved interior surface 30 has at least one drain hole or a first drain hole and a second drain hole 38 and 40. The drain holes 38 and 40 are a system installed to improve ground stability and drainage as the device 10 provides the moisture or material fluid on the handle bar 12. The device 10 further includes a cap 42 that requires a special tool to unlock the cap 42 to disassemble the upper housing 16.

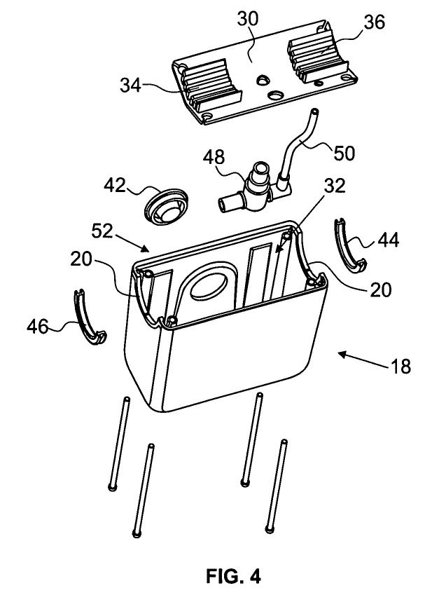

Turning now to FIG. 4, there is shown a first and a second curved member 44 and 46 that rest in respective apertures 20 and 20 of the lower housing 18. The first and the second curved members 44 and 46 are preferably each a flat, smooth rubber blade, used to remove or control the flow of liquid on a surface of the handle bar 12. Preferably, the first and the second curved members 44 and 46 are squeegee type members and push the fluid applied on disposed on the handle bar 12 in the same direction that the lower housing 18 is moved. Although shown as a curved member 44 and 46, the members may be differently shaped. Curved members 44 and 46 may also comprise silicone in another embodiment.

Disposed within the interior space 32 is preferably a pump 48 that is connected to a liquid tube 50. Pump 48 is preferably connected to a receptacle 52 that contains a cleaning fluid. Pump 48 draws the cleaning fluid from the receptacle 52 and supplies the liquid to the liquid tube 50. Liquid tube 50 supplies the liquid to the handle bar 12. The pump 48 in one embodiment is a generic squirt gun type plastic pump with a fitted piston (not shown) that is spring loaded via a spring (not shown), and that has at least two ball check valves (not shown). Rod from push button forces piston up and down to spray sanitizer above handle 12.

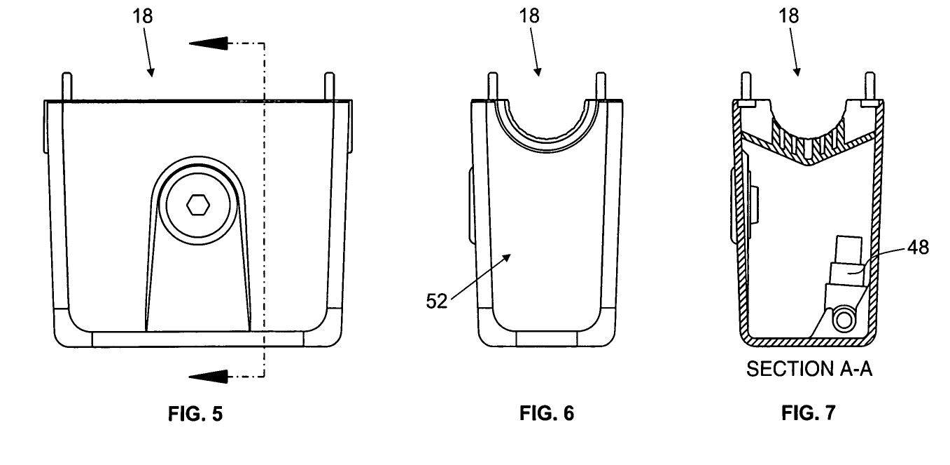

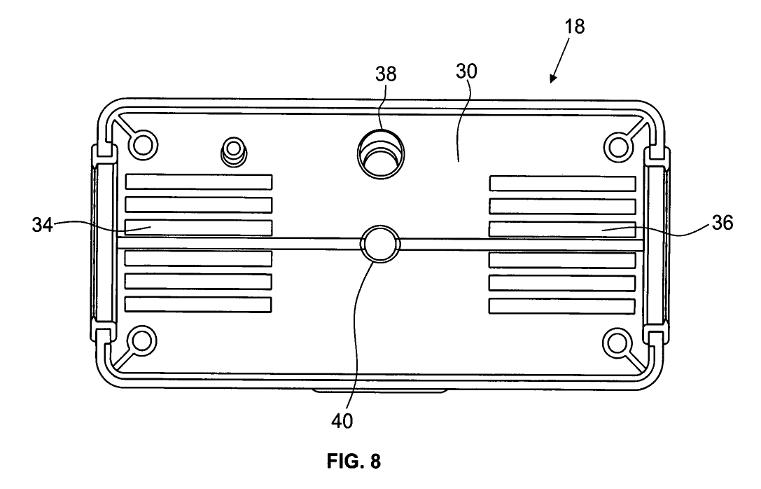

FIGS. 5-7 show a first, second and third view of the lower housing 18. The lower housing 18 is shown in FIG. 6 as having a liquid tank 52 on one side of the lower housing 18. FIG. 7 shows a cross sectional view of the lower housing 18 of FIG. 5 along line A-A. FIG. 7 shows the pump 48 disposed on an opposite lateral side relative to the tank 52 and within the lower housing 18.

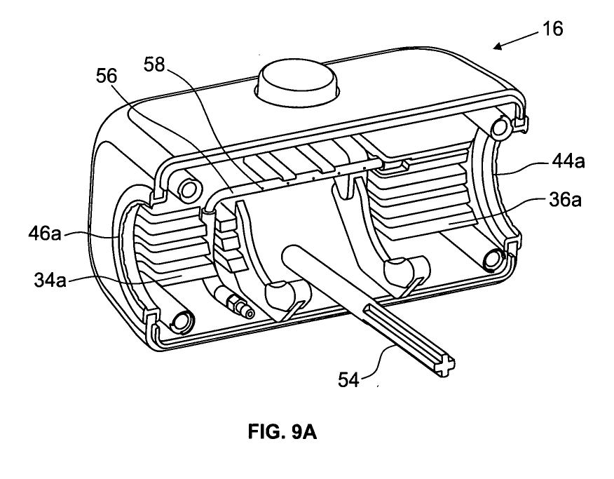

FIG. 8 shows a bottom view of the upper lower housing 18 having the first and the second slide rails 34 and 36 and the first and second drain holes 38 and 40 disposed through the curved surface 30. As can be seen the first drain hole 38 is shown. The device 10 also has a second hole 40. The second hole 40 forms a return drain hole 40 that is disposed through the surface 30 and is located underneath the handle bar 12.

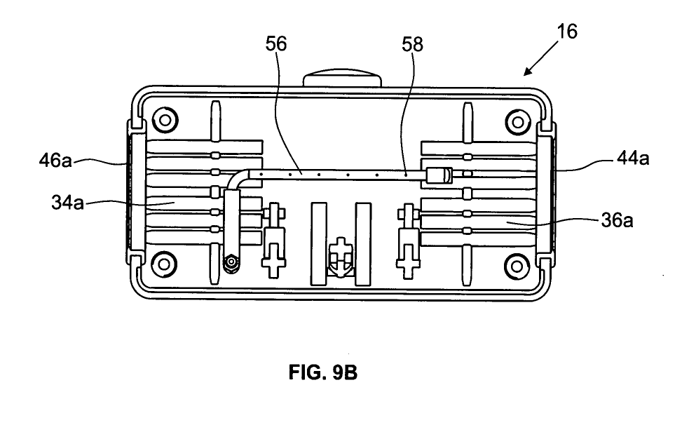

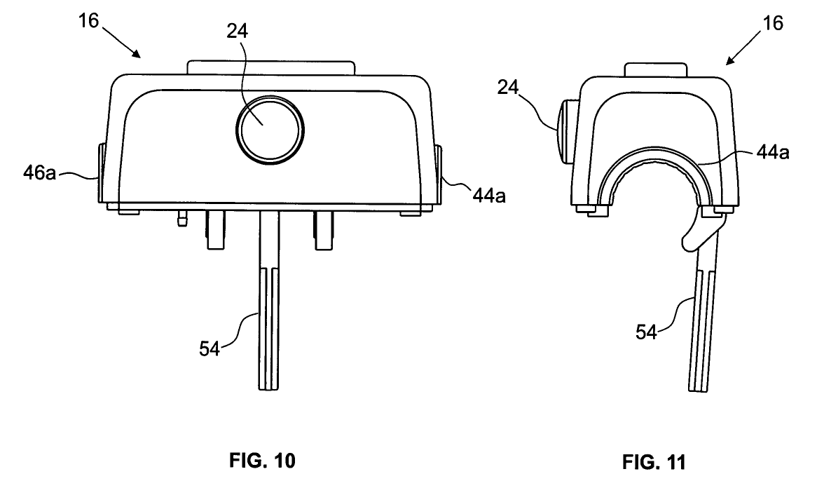

Now, the upper housing member 16 will be discussed with the components therein to apply a solution to the handle bar 12 and also to clean the handle bar 12 and also to support the upper housing member 16 on the handle bar 12. FIGS. 9A and 9B show the assembled upper housing member 16 having the first and the second slide rails 34a and 36a and a pump rod 54. The pump rod 54 extends from the upper housing 16 and into the lower housing 18 Preferably a tubular member 56 is provided that is generally parallel disposed to the curved interior surface 30 of the lower housing 18 and the handle bar 12, when assembled and placed on the handle bar 12. The tubular member 56 includes a number of apertures 58 thereon. Preferably, the solution is drawn from the pump 48 and the pump rod 54 from the receptacle 52 and introduced to the tubular member 56. The tubular member 56 has the solution within the tubular member 56 and releases the solution over the handle bar 12 via the apertures 58 thereon. The solution disposed on the handle bar 12 preferably cleans the handle bar's 12 surface. Handle 12 is mostly cleaned preferably by spraying above the handle 12 through pin holes in the tubular member 56. It should be appreciated that in another embodiment one or more round seals may need slits to direct liquid around the handle 12 diameter.

The upper housing 16 preferably also has a first and a second slide rails 34a and 36a that are disposed within the upper housing 16. The first and the second slide rails 34a and 36a preferably are curved members that fit on the handle bar 12 and traverse over the handle bar 12. The first and second slide rails 34a and 36a preferably form a way track or, more particularly a track or surface structure that supports and guides the device 10 on the handle bar 12 along with the first and the second slide rails 34 and 36 of the lower housing 18.

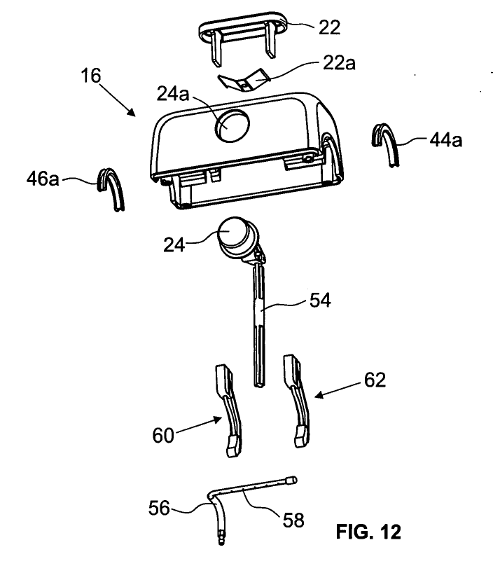

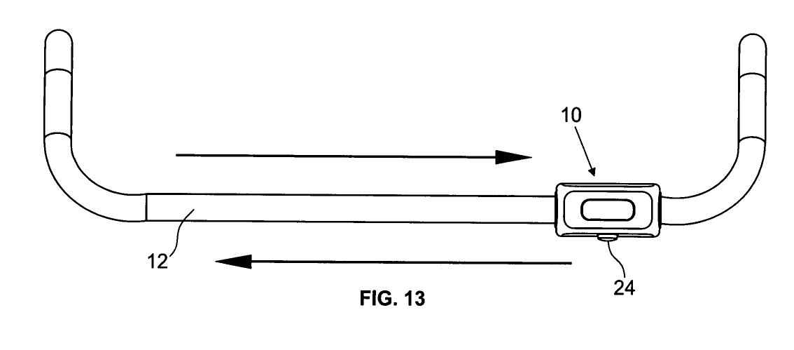

FIGS. 10 and 11 show the upper housing 16. The upper housing 16 has the pump rod 54 and first and second curved members 44a and 46a. The curved members 44a and 46a are disposed on an arc or on a portion of the apertures 20 and 20. The first and the second curved member 44a and 46a preferably rest in or near respective apertures 20 and 20 of the upper housing 16. The first and the second curved members 44a and 46a are preferably each a flat, smooth rubber or silicone blade, used to remove or control the flow of liquid on a surface of the handle bar 12 along with the first and the second curved members 44a and 46a of the lower housing 18 as shown in FIG. 12. Preferably, the first and the second curved members 44a and 46a are squeegee type members and push the fluid applied on disposed on the handle bar 12 in the same direction that the sanitizing device 10 is moved. Although shown as a curved member 44a and 46a, the members 44a and 46a may be differently shaped. The upper housing 16 further includes a button 24 to release the fluid and that is operatively connected to drawn and release the liquid from the receptacle 52.

A button spring 22a is provided underneath the button 22 as shown in FIG. 12. Preferably, the button 22 actuates the members 60 and 62 to release and open the upper and the lower housings 16 and 18. The upper housing member 16 also has a liquid spray tube 56 with the apertures 58 to release the fluid operatively connected to the pump 48. Preferably, the button 24 forms a portion of the pump rod 54 and fits through aperture 24a shown in the upper housing 16. Two soft rubber brake pads on arms are shown as elements 60 and 62. Members 60 and 62 preferably grab cart handle 12 until the user pushes down on the unlock button 22.

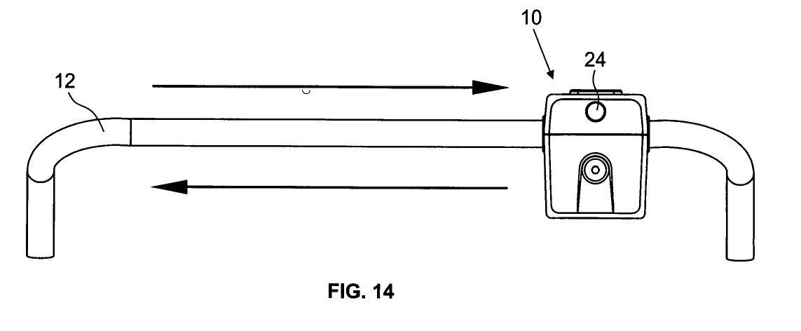

As shown in FIGS. 13 and 14, the sanitizing device 10 may traverse over the handle bar 12. The user may press button 24. The button 24 actuates the pump rod 54 connected to the pump 54, which release fluid from the receptacle 52, and which is communicated to the tube 56 via apertures 58. The tubular member 56 applies solution from the liquid spray tube and tubular member 58 and applies the solution to clean the handle bar 12 as shown, which is stationary. Thereafter, as the device 10 is moved from side to side the squeegee members 24 and 24a and 26 and 26a direct the fluid to a respective right or left direction depending on the user's movement of the device 10 to clean and dry the handle bar 12. Solution can be alcohol, cleaning solution, vinegar, PINESOL®, WINDEX®, soap and water, CLOROX®, PURELL® Instant Hand Sanitizer, hand sanitizer, or any other liquid or solid material that may clean, sanitize, or disinfect the handle bar 12. In another embodiment, the device 10 may not use a liquid at all and may have a heater (not shown) to heat and clean the surface of the handle 12. It should be understood, of course, that the foregoing relates to exemplary embodiments of the invention and that modifications may be made without departing from the spirit and scope of the invention as set forth in the following claims.

Furthermore, combinations of embodiments of the invention may be divided into specific functions and implemented on different individual computer processing devices and systems which may be interconnected to communicate and interact with each other. Dividing up the functionality of the invention between several different computers is meant to be covered within the scope of the invention.

While this invention has been particularly shown and described with references to a preferred embodiment thereof, it will be understood by those skilled in the art that is made therein without departing from the spirit and scope of the invention as defined by the following claims.

What is claimed is:

1. A sanitizing device for a handle bar, said device having an elongated axis, said device comprising:
a housing unit for mounting on the handle bar, said housing fitting around said handle bar;
a reservoir inside said housing unit for storing sanitizing materials; and
a dispenser inside said housing unit, said dispenser connected to said reservoir for dispensing sanitizing materials from said reservoir to said handle bar.

2. The sanitizing device of claim 1, further comprising an aperture defined within the housing unit to permit the handle bar to traverse through the housing unit.

3. The sanitizing device of claim 1, further comprising a member made with a second material having an edge, wherein the member is drawn across a surface of the handle bar to remove the sanitized material deposited thereon.

4. The sanitizing device of claim 1, further comprising a pump to draw the sanitized material from the reservoir and to the handle bar.

5. The sanitizing device of claim 1, further comprising a first and a second aperture defined on the housing unit to allow the handle bar to access the housing unit; and further comprising a surface generally parallel to the handle bar, wherein the surface has a slide rail disposed thereon to contact the handle bar.

6. The sanitizing device of claim 5, further comprising a second slide rail disposed on the surface to contact the handle bar.

7. The sanitizing device of claim 6, further comprising at least one drain hole to collect sanitizing material from the handle bar, the drain hole being formed on the surface.

8. The sanitizing device of claim 1, wherein the housing unit comprises a first housing member and a second housing member.

9. The sanitizing device of claim 8, further comprising an elongated tube comprises a plurality of apertures, the elongated tube being disposed under the first housing member and generally over the handle bar, the elongated tube providing the sanitizing material to the handle bar through the plurality of apertures.

10. The sanitizing device of claim 8, further comprising a first aperture for receiving the handle bar, and a second aperture on an opposite side of the housing unit relative to the first aperture, and further comprising a first member disposed around the first aperture made with a second material having an edge, wherein the first member is drawn across a surface of the handle bar to remove the sanitized material deposited thereon.

11. The sanitizing device of claim 10, further comprising a second member disposed around the second aperture made with the second material and having the edge, wherein the second member is drawn across the surface of the handle bar to remove the sanitized material deposited thereon.

12. The sanitizing device of claim 1, further comprising a first input being connected to a pump, wherein when the first input is actuated the sanitized material is released onto the handle bar.

13. The sanitizing device of claim 12, wherein the housing unit comprises at least two members, and further comprising a second input being different from the first input, wherein when the second input is actuated the housing unit is opened.

14. The sanitizing device of claim 1, wherein the sanitizing material comprising a fluid.

15. The sanitizing device of claim 14, wherein the sanitizing material is recaptured via at least one drain hole and directed into a said reservoir.

16. A method of cleaning a handle bar comprising:
fitting a device around the handle bar;
dispensing a material from the device to the handle bar; and
moving the device on the handle bar with a second material connected to the device to clean the handle bar with the second material.

17. The method of claim 16, further comprising move the device on the handle bar with the second material comprising a squeegee material connected to the device.

18. The method of claim 16, further comprising fitting the device completely around the handle bar.

19. The method of claim 16, further comprising dispensing the material from within an inside of the device to the handle bar.

20. A cleaning device for a handle bar comprising:
a housing completely surrounding the handle bar;
a nozzle for dispensing a cleaning fluid to the handle bar, the nozzle being located within the housing;
the housing comprising a first aperture and a second aperture, the first aperture and the second aperture being on opposite lateral sides of the housing; and
a squeegee material surrounding the first aperture and the second aperture.
